(19) United States
(12) Patent Application Publication
(10) Pub. No.: US 2008/0139819 A1
(43) Pub. Date: Jun. 12, 2008

(54) Process for the Synthesis of Monosulfated Derivatives of Substituted Benzoxazoles

(75) Inventors: Youchu WANG, Sainte-Anne-de-Bellevue (CA); Silvio IERA, Montreal (CA); Maria PAPAMICHELAKIS, Montreal (CA)

Correspondence Address: Pepper Hamilton LLP/Wyeth, 400 Berwyn Park, 899 Cassatt Road, Berwyn, PA, 19312-1183, US

(73) Assignee: WYETH, Madison, NJ (US)

(21) Appl. No.: 11/947,057

(22) Filed: Nov. 29, 2007

Related U.S. Application Data
(60) Provisional application No. 60/867,866, filed on Nov. 30, 2006.

(51) Int. Cl. C07D 263/56

(52) U.S. Cl. 548/224

(57) ABSTRACT

The present invention provides synthetic processes for the preparation of mono-sulfated derivatives of substituted benzoxazoles, which are useful as estrogenic agents.

This application claims benefit of priority to U.S. provisional patent application Ser. No. 60/867,866 filed Nov. 30, 2006, which is hereby incorporated in its entirety.

### FIELD OF THE INVENTION

This invention relates to processes for the preparation of mono-sulfated derivatives of substituted benzoxazoles, which are useful as estrogenic agents.

### BACKGROUND OF THE INVENTION

The pleiotropic effects of estrogens in mammalian tissues have been well documented, and it is now appreciated that estrogens affect many organ systems [Mendelsohn and Karas, New England Journal of Medicine 340: 1801-1811 (1999), Epperson, et al., Psychosomatic Medicine 61: 676-697 (1999), Crandall, Journal of Womens Health & Gender Based Medicine 8: 1155-1166 (1999), Monk and Brodaty, Dementia & Geriatric Cognitive Disorders 11: 1-10 (2000), Hurn and Macrae, Journal of Cerebral Blood Flow & Metabolism 20: 631-652 (2000), Calvin, Maturitas 34: 195-210 (2000), Finking, et al., Zeitschrift fur Kardiologie 89: 442-453 (2000), Brincat, Maturitas 35: 107-117 (2000), Al-Azzawi, Postgraduate Medical Journal 77: 292-304 (2001)]. Estrogens can exert effects on tissues in several ways, and the most well characterized mechanism of action is their interaction with estrogen receptors leading to alterations in gene transcription. Estrogen receptors are ligand-activated transcription factors and belong to the nuclear hormone receptor superfamily. Other members of this family include the progesterone, androgen, glucocorticoid and mineralocorticoid receptors. Upon binding ligand, these receptors dimerize and can activate gene transcription either by directly binding to specific sequences on DNA (known as response elements) or by interacting with other transcription factors (such as AP1), which in turn bind directly to specific DNA sequences [Moggs and Orphanides, EMBO Reports 2: 775-781 (2001), Hall, et al., Journal of Biological Chemistry 276: 36869-36872 (2001), McDonnell, Principles Of Molecular Regulation. p 351-361 (2000)]. A class of “coregulatory” proteins can also interact with the ligand-bound receptor and further modulate its transcriptional activity [McKenna, et al., Endocrine Reviews 20: 321-344 (1999)]. It has also been shown that estrogen receptors can suppress NFκB-mediated transcription in both a ligand-dependent and independent manner [Quaedackers, et al., Endocrinology 142: 1156-1166 (2001), Bhat, et al., Journal of Steroid Biochemistry & Molecular Biology 67: 233-240 (1998), Pelzer, et al., Biochemical & Biophysical Research Communications 286: 1153-7 (2001)].

Estrogen receptors can also be activated by phosphorylation. This phosphorylation is mediated by growth factors such as EGF and causes changes in gene transcription in the absence of ligand [Moggs and Orphanides, EMBO Reports 2: 775-781 (2001), Hall, et al., Journal of Biological Chemistry 276: 36869-36872 (2001)].

A less well-characterized means by which estrogens can affect cells is through a so-called membrane receptor. The existence of such a receptor is controversial, but it has been well documented that estrogens can elicit very rapid non-genomic responses from cells. The molecular entity responsible for transducing these effects has not been definitively isolated, but there is evidence to suggest it is at least related to the nuclear forms of the estrogen receptors [Levin, Journal of Applied Physiology 91: 1860-1867 (2001), Levin, Trends in Endocrinology & Metabolism 10: 374-377 (1999)].

Two estrogen receptors have been discovered to date. The first estrogen receptor was cloned about 15 years ago and is now referred to as ERα [Green, et al., Nature 320: 134-9 (1986)]. The second form of the estrogen receptor was found comparatively recently and is called ERβ [Kuiper, et al., Proceedings of the National Academy of Sciences of the United States of America 93: 5925-5930 (1996)]. Early work on ERβ focused on defining its affinity for a variety of ligands and indeed, some differences with ERα were seen. The tissue distribution of ERβ has been well mapped in the rodent and it is not coincident with ERα. Tissues such as the mouse and rat uterus express predominantly ERα, whereas the mouse and rat lung express predominantly ERβ [Couse, et al., Endocrinology 138: 4613-4621 (1997), Kuiper, et al., Endocrinology 138: 863-870 (1997)]. Even within the same organ, the distribution of ERα and ERβ can be compartmentalized. For example, in the mouse ovary, ERβ is highly expressed in the granulosa cells and ERα is restricted to the thecal and stromal cells [Sar and Welsch, Endocrinology 140: 963-971 (1999), Fitzpatrick, et al., Endocrinology 140: 2581-2591 (1999)]. However, there are examples where the receptors are coexpressed and there is evidence from in vitro studies that ERα and ERβ can form heterodimers [Cowley, et al., Journal of Biological Chemistry 272: 19858-19862 (1997)].

A large number of compounds have been described that either mimic or block the activity of 17β-estradiol. Compounds having roughly the same biological effects as 17β-estradiol, the most potent endogenous estrogen, are referred to as “estrogen receptor agonists”. Those which, when given in combination with 17β-estradiol, block its effects are called “estrogen receptor antagonists”. In reality there is a continuum between estrogen receptor agonist and estrogen receptor antagonist activity and indeed some compounds behave as estrogen receptor agonists in some tissues and estrogen receptor antagonists in others. These compounds with mixed activity are called selective estrogen receptor modulators (SERMS) and are therapeutically useful agents (e.g. EVISTA) [McDonnell, Journal of the Society for Gynecologic Investigation 7: S10-S15 (2000), Goldstein, et al., Human Reproduction Update 6: 212-224 (2000)]. The precise reason why the same compound can have cell-specific effects has not been elucidated, but the differences in receptor conformation and/or in the milieu of coregulatory proteins have been suggested.

It has been known for some time that estrogen receptors adopt different conformations when binding ligands. However, the consequence and subtlety of these changes has been only recently revealed. The three dimensional structures of ERα and ERβ have been solved by co-crystallization with various ligands and clearly show the repositioning of helix 12 in the presence of an estrogen receptor antagonist which sterically hinders the protein sequences required for receptor-coregulatory protein interaction [Pike, et al., Embo 18: 4608-4618 (1999), Shiau, et al., Cell 95: 927-937 (1998)]. In addition, the technique of phage display has been used to identify peptides that interact with estrogen receptors in the presence of different ligands [Paige, et al., Proceedings of the National Academy of Sciences of the United States of America 96: 3999-4004 (1999)]. For example, a peptide was identified that distinguished between ERα bound to the full estrogen receptor agonists 17β-estradiol and diethylstilbesterol. A different peptide was shown to distinguish between clomiphene bound to ERα and ERβ. These data indicate that each ligand potentially places the receptor in a unique and unpredictable conformation that is likely to have distinct biological activities.

As mentioned above, estrogens affect a panoply of biological processes. In addition, where gender differences have been described (e.g. disease frequencies, responses to challenge, etc), it is possible that the explanation involves the difference in estrogen levels between males and females.

Compounds having estrogenic activity are disclosed in U.S. Pat. No. 6,794,403, which is incorporated herein by reference in its entirety. Given the importance of these compounds, it can be seen that a continuing need exists for new processes for their preparation. This invention is directed to these, as well as other, important ends.

### SUMMARY OF THE INVENTION

In some embodiments, the present invention provides processes for the preparation of mono-sulfated derivatives of substituted benzoxazoles, which are useful as estrogenic agents. In some embodiments, the invention provides synthetic processes comprising:

reacting (sulfating) a compound of Formula II:

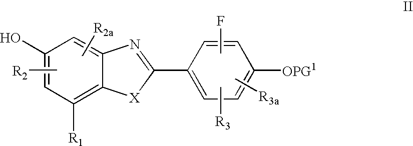

or a salt thereof, wherein:
PG¹ is a hydroxyl protecting group;
R₁ is hydrogen, hydroxyl, halogen, alkyl of 1-6 carbon atoms, trifluoroalkyl of 1-6 carbon atoms, cycloalkyl of 3-8 carbon atoms, alkoxy of 1-6 carbon atoms, trifluoroalkoxy of 1-6 carbon atoms, thioalkyl of 1-6 carbon atoms, sulfoxoalkyl of 1-6 carbon atoms, sulfonoalkyl of 1-6 carbon atoms, aryl of 6-10 carbon atoms, a 5 or 6-membered heterocyclic ring having 1 to 4 heteroatoms selected from O, N or S, —NO₂, —NR₅R₆, —N(R₅)COR₆, —CN, —CHFCN, —CF₂CN, alkynyl of 2-7 carbon atoms, or alkenyl of 2-7 carbon atoms; wherein the alkyl or alkenyl moieties are optionally substituted with hydroxyl, —CN, halogen, trifluoroalkyl, trifluoroalkoxy, —COR₅, —CO₂R₅, —NO₂, CONR₅R₆, NR₅R₆ or N(R₅)COR₆;
R₂ and R₂ₐ are each, independently, hydrogen, hydroxyl, halogen, alkyl of 1-6 carbon atoms, alkoxy of 1-4 carbon atoms, alkenyl of 2-7 carbon atoms, or alkynyl of 2-7 carbon atoms, trifluoroalkyl of 1-6 carbon atoms, or trifluoroalkoxy of 1-6 carbon atoms; wherein the alkyl or alkenyl moieties are optionally substituted with hydroxyl, —CN, halogen, trifluoroalkyl, trifluoroalkoxy, —COR₅, —CO₂R₅, —NO₂, CONR₅R₆, NR₅R₆ or N(R₅)COR₆;
R₃, R₃ₐ, and R₄ are each, independently, hydrogen, alkyl of 1-6 carbon atoms, alkenyl of 2-7 carbon atoms, alkynyl of 2-7 carbon atoms, halogen, alkoxy of 1-4 carbon atoms, trifluoroalkyl of 1-6 carbon atoms, or trifluoroalkoxy of 1-6 carbon atoms; wherein the alkyl or alkenyl moieties are optionally substituted with hydroxyl, —CN, halogen, trifluoroalkyl, trifluoroalkoxy, —COR₅, —CO₂R₅, —NO₂, CONR₅R₆, NR₅R₆ or N(R₅)COR₆;
R₅, R₆ are each, independently hydrogen, alkyl of 1-6 carbon atoms, aryl of 6-10 carbon atoms;
X is O, S, or NR₇; and
R₇ is hydrogen, alkyl of 1-6 carbon atoms, aryl of 6-10 carbon atoms, —COR₅, —CO₂R₅ or —SO₂R₅,

with a sulfating reagent, for a time and under conditions sufficient to form a compound of Formula I or Ia:

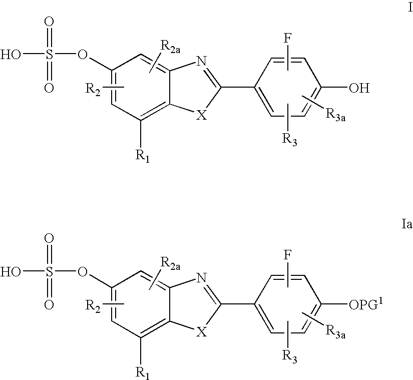

or a salt thereof.

In some embodiments, the processes further include isolating a salt of the compound of Formula I, wherein the salt has Formula Ib:

[R¹⁰—O—SO₃⁻¹]_{q}M Ib

wherein:

R¹⁰ is:

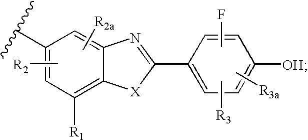

M is a Group I or II metal ion; and

q is 1 when M is Group I metal ion, or q is 2 when M is a Group II metal ion.

In some further embodiments, the processes further include reacting a compound of Formula III:

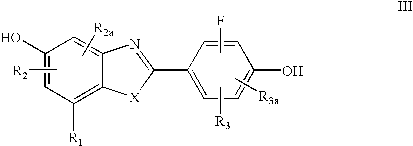

or a salt thereof, with a hydroxyl protecting group reagent for a time and under conditions sufficient to form the compound of Formula II.

In some embodiments, the processes further include isolating the formed compound of Formula II or salt thereof.

In some preferred embodiments, the compound of Formula II has the structure of Formula II-1:

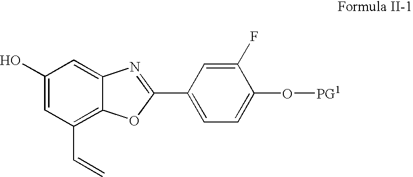

or is a salt thereof;

the compound of Formula I has the structure of Formula I-1:

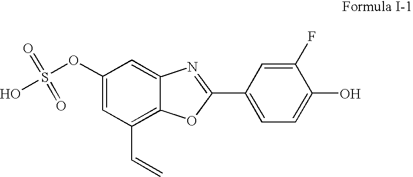

or is a salt thereof; and

the compound of Formula Ia has the structure of Formula Ia-1:

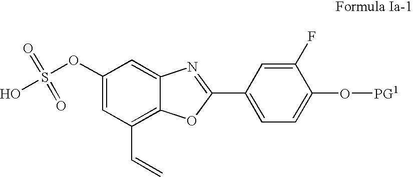

or is a salt thereof.

In some preferred embodiments, R¹⁰ has the structure of Formula R^{10a}:

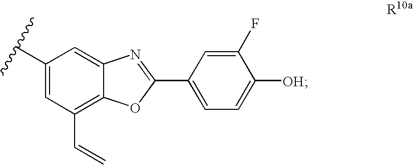

and the compound of Formula III has the structure of Formula IIIa:

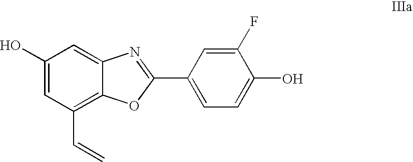

or is a salt thereof.

### DESCRIPTION OF THE INVENTION

In some embodiments, the invention provides synthetic processes comprising:

reacting (sulfating) a compound of Formula II:

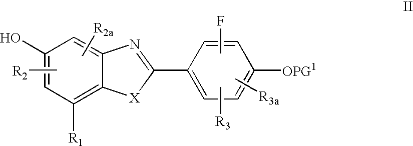

or a salt thereof, wherein:
PG¹ is a hydroxyl protecting group;
R₁ is hydrogen, hydroxyl, halogen, alkyl of 1-6 carbon atoms, trifluoroalkyl of 1-6 carbon atoms, cycloalkyl of 3-8 carbon atoms, alkoxy of 1-6 carbon atoms, trifluoroalkoxy of 1-6 carbon atoms, thioalkyl of 1-6 carbon atoms, sulfoxoalkyl of 1-6 carbon atoms, sulfonoalkyl of 1-6 carbon atoms, aryl of 6-10 carbon atoms, a 5 or 6-membered heterocyclic ring having 1 to 4 heteroatoms selected from O, N or S, —NO₂, —NR₅R₆, —N(R₅)COR₆, —CN, —CHFCN, —CF₂CN, alkynyl of 2-7 carbon atoms, or alkenyl of 2-7 carbon atoms; wherein the alkyl or alkenyl moieties are optionally substituted with hydroxyl, —CN, halogen, trifluoroalkyl, trifluoroalkoxy, —COR₅, —CO₂R₅, —NO₂, CONR₅R₆, NR₅R₆ or N(R₅)COR₆;
R₂ and R₂ₐ are each, independently, hydrogen, hydroxyl, halogen, alkyl of 1-6 carbon atoms, alkoxy of 1-4 carbon atoms, alkenyl of 2-7 carbon atoms, or alkynyl of 2-7 carbon atoms, trifluoroalkyl of 1-6 carbon atoms, or trifluoroalkoxy of 1-6 carbon atoms; wherein the alkyl or alkenyl moieties are optionally substituted with hydroxyl, —CN, halogen, trifluoroalkyl, trifluoroalkoxy, —COR₅, —CO₂R₅, —NO₂, CONR₅R₆, NR₅R₆ or N(R₅)COR₆;
R₃, R₃ₐ, and R₄ are each, independently, hydrogen, alkyl of 1-6 carbon atoms, alkenyl of 2-7 carbon atoms, alkynyl of 2-7 carbon atoms, halogen, alkoxy of 1-4 carbon atoms, trifluoroalkyl of 1-6 carbon atoms, or trifluoroalkoxy of 1-6 carbon atoms; wherein the alkyl or alkenyl moieties are optionally substituted with hydroxyl, —CN, halogen, trifluoroalkyl, trifluoroalkoxy, —COR₅, —CO₂R₅, —NO₂, CONR₅R₆, NR₅R₆ or N(R₅)COR₆;
R₅, R₆ are each, independently hydrogen, alkyl of 1-6 carbon atoms, aryl of 6-10 carbon atoms;
X is O, S, or NR₇; and
R₇ is hydrogen, alkyl of 1-6 carbon atoms, aryl of 6-10 carbon atoms, —COR₅, —CO₂R₅ or —SO₂R₅,

with a sulfating reagent, for a time and under conditions sufficient to form a compound of Formula I or Ia:

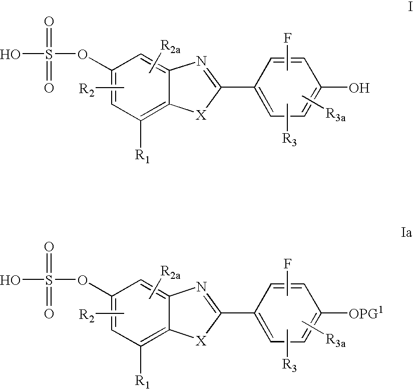

or a salt thereof, or a mixture thereof.

In some embodiments, the present processes are used to prepare compounds of Formula I or Ia or salts thereof that are substantially free of compounds of Formula X or Xa:

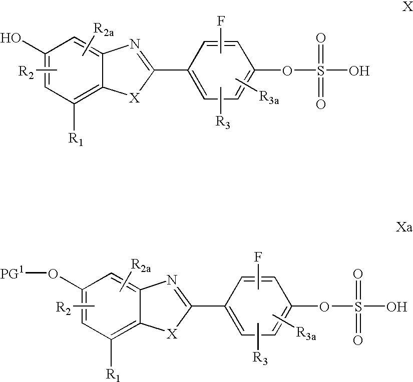

or salts thereof, wherein R₁, R₂, R₂ₐ, R₃, R₃ₐ, PG¹ and X are the same as defined in the compounds Formula I and Ia herein. As used herein, the term “substantially free of compounds of Formula X or Xa” means that no more than about 5% by weight, preferably no more than about 2% by weight, more preferably no more that about 1% by weight, and more preferably no more than about 0.5% by weight of a given sample of compound has Formula X or Xa or a salt thereof.

A general outline of some embodiments of the processes of the present invention is provided in Scheme I below:

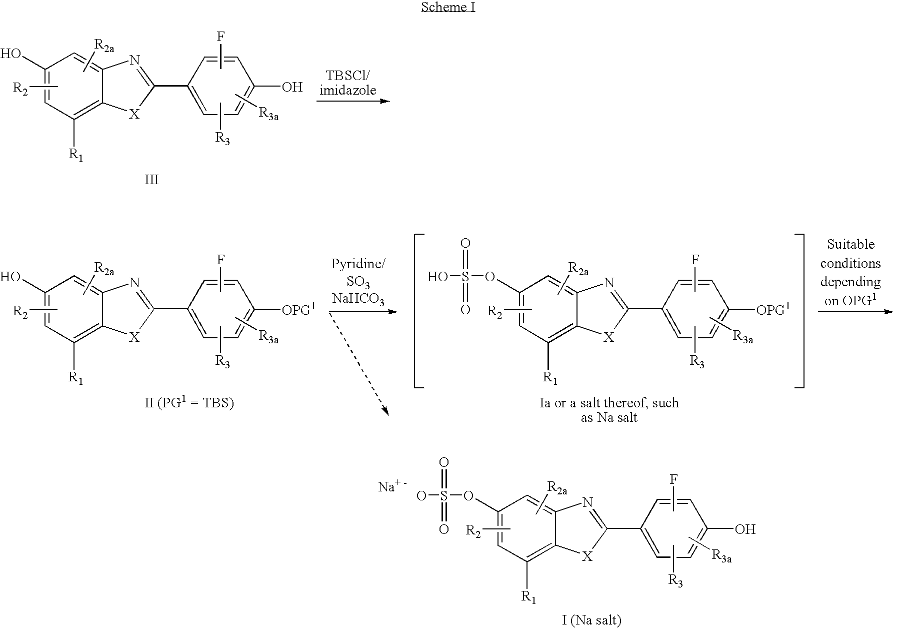

The preparation of compounds of Formula III is described in U.S. Pat. No. 6,794,403, incorporated by reference herein in its entirety.

It will be appreciated that the starting material of Formula III has two reactive hydroxyl groups and the present invention surprisingly provides a convenient route for the preparation of the mono-sulfated product of Formula I which is substantially free of di-sulfated by-product and is substantially free of the product of formula X below which is mono-sulfated at the phenolic hydroxyl group.

In some preferred embodiments, the compound of Formula II has the structure of Formula II-1:

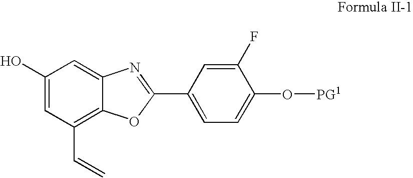

or is a salt thereof;

the compound of Formula I has the structure of Formula I-1:

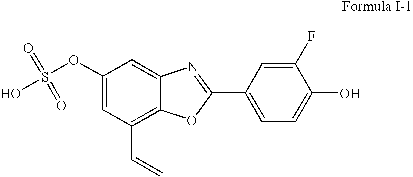

or is a salt thereof; and

the compound of Formula Ia has the structure of Formula Ia-1:

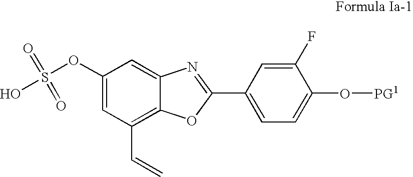

or a salt thereof.

As can be seen in Scheme I, the compound of Formula III is selectively protected at the phenyl hydroxyl thereof, and then sulfated at the unprotected (e.g., benzoxazole where X═O) hydroxyl to afford the compound of Formula Ia or a salt thereof. In some embodiments, during the workup of isolating the compound of Formula Ia or a salt thereof from the reaction mixture, the workup conditions are sufficient to remove the protecting group —PG¹ of the compound of Formula Ia or a salt thereof to afford the compound of formula I or a salt thereof.

The protecting group is added by reacting the compound of Formula III (such as the compound of Formula IIIa) with a hydroxyl protecting group reagent, which in some embodiments has the structure PG¹-Q, where PG¹ is a protecting group, and Q is a leaving group that is displaced by the phenolic oxygen atom attached to the 4-position of the phenyl ring of the compound of Formula III. Without wishing to be bound by a particular theory, it is believed that the phenolic hydroxyl (attached to the phenyl ring of the compound of Formula II) is more acidic than the hydroxyl attached to the fused benzene ring, thus preferentially affording reaction of the phenolic hydroxyl with the hydroxyl protecting group reagent. In some embodiments, less than about one molar equivalent of the hydroxyl protecting group reagent is used in the reaction. In some embodiments, about one molar equivalent of the hydroxyl protecting group reagent is used in the reaction. When about one molar equivalent, or less, of a base is added to the reaction mixture that includes compound of Formula II, the phenolic hydroxyl group is preferentially deprotonated, thus generating phenoxide anion. The phenoxide anion reacts with the hydroxyl protecting group reagent more readily than does the hydroxyl attached to the fused benzene ring of the bi-cyclic ring of the compound of Formula III.

Suitable hydroxyl protecting groups include those having the structure —SiR^{a}R^{b}R^{c}; wherein R^{a}, R^{b} and R^{c} are each, independently, C₁₋₆ alkyl. One preferred hydroxyl protecting group is tert-butyldimethylsilyl (TBS), which can be attached to the phenolic hydroxyl of the compound of Formula III by reaction with the hydroxyl protecting group reagent tert-butyldimethylsilyl chloride. In some embodiments, the hydroxyl protecting group reagent, for example tert-butyldimethylsilyl chloride, is employed in an amount that is at least about one molar equivalent to the compound of Formula III. Other suitable hydroxyl protecting groups and hydroxyl protecting group reagents are disclosed in Greene and Wuts, Protective Groups in Organic Synthesis, 2d ed, John Wiley & Sons, New York, 1991, the disclosure of which is incorporated herein by reference in its entirety.

In some embodiments, a base is present in the reaction between the compound of Formula III and the hydroxyl protecting group reagent. In some embodiments, the amount of the base is less than 1.1 molar equivalents to that of the compound of Formula III. In some embodiments, the amount of the base is less than 1.05 molar equivalents to that of the compound of Formula III. In some embodiments, the amount of the base is less than 1.02 molar equivalents to that of the compound of Formula III. In some embodiments, the amount of the base is less than 1.01 molar equivalents to that of the compound of Formula II. In some embodiments, the amount of the base is less than about one molar equivalent to the compound of Formula II. In some embodiments, the amount of the base is about 0.95 to about 1.02 molar equivalents to that of the compound of Formula II. In some embodiments, the amount of the base is about 0.98 to about 1.01 molar equivalents to that of the compound of Formula III. In some embodiments, the amount of the base is about 0.99- to about 1.0 molar equivalents to that of the compound of Formula III. Preferably, the base is present in an amount that is about one molar equivalent to the compound of Formula III. It is believed that less than about one molar equivalent of the base (relevant to the compound of Formula II) is used to minimize deprotonation of the hydroxyl attached to the fused benzene ring of the bi-cyclic ring of the compound of Formula III. In such situation, the hydroxyl protecting group reagent preferentially reacts with the deprotonated phenolic hydroxyl group (over the reaction with the hydroxyl attached to the fused benzene ring of the bi-cyclic ring of the compound of Formula II). A wide variety of bases can be employed. In some embodiments, the base is an amine. Suitable amines include acyclic amines such as alkylamines (for example, trialkylamines including triethylamine and trimethylamine), dimethylphenylamine and dimethylbenzylamine; cyclic amines (for example, pyrrolidine, piperidine, 1-methylpyrrolidine and 1-methylpiperidine); and aromatic amines (which have one or more nitrogen atoms as ring-forming atoms of the aromatic ring, for example, imidazole, pyridine and pyrimidine). In some embodiments, the base is an aromatic amine, for example, imidazole.

Typically, the reaction of the compound of Formula III and the hydroxyl protecting group reagent is performed in a solvent system, that can be a single solvent, or can include a mixture of solvents. A wide variety of suitable solvents can be employed, including polar organic solvents, preferably polar aprotic organic solvents—i.e., organic solvents that are not readily deprotonated in the presence of a strongly basic reactant or reagent. Suitable aprotic solvents can include, by way of example and without limitation, ethers (e.g., tetrahydrofuran), dimethylformamide (DMF), dimethylacetamide (DMAC), 1,3-dimethyl-3,4,5,6-tetrahydro-2(1H)-pyrimidinone (DMPU), 1,3-dimethyl-2-imidazolidinone (DMI), N-methylpyrrolidinone (NMP), formamide, N-methylacetamide, N-methylformamide, acetonitrile, dimethyl sulfoxide (DMSO), propionitrile, ethyl formate, methyl acetate, hexachloroacetone, ethyl acetate, sulfolane, N,N-dimethylpropionamide, tetramethylurea, nitrobenzene, or hexamethylphosphoramide. Also included within the term aprotic solvent are esters, hydrocarbons, and many ether solvents including: dimethoxymethane, tetrahydrofuran, 1,3-dioxane, 1,4-dioxane, furan, diethyl ether, tetrahydropyran, diisopropyl ether, dibutyl ether, ethylene glycol dimethyl ether, ethylene glycol diethyl ether, diethylene glycol dimethyl ether, diethylene glycol diethyl ether, triethylene glycol dimethyl ether, anisole, and t-butyl methyl ether. In some embodiments, the reaction is performed in a solvent system that includes or consists of an ether, for example tetrahydrofuran.

Typically, the compound of Formula III and the base are added to the solvent system at a suitable temperature (for example room temperature), and the solution is then cooled, for example to a temperature less than about 10° C., preferably between about −10° C. and 10° C., for example about 0° C., prior to the addition of the hydroxyl protecting group reagent. The progress of the reaction can be monitored by a variety of techniques, for example by chromatographic techniques. Typically, the reaction between the compound of Formula III and the hydroxyl protecting group reagent is complete after about 18 hours to about 2 days. When the reaction is complete, the protected compound of Formula II, or salt thereof, can be isolated form the reaction mixture by standard work-up procedures, for example by filtering the reaction mixture, evaporating the residue. If desired, the product can be then be purified by any standard technique, for example by flash chromatography over silica. Generally, it is preferable to perform the purification prior to the next step.

In some embodiments, compounds of Formula II or salts thereof prepared by the present processes are substantially free of compounds of Formula XX or XXa:

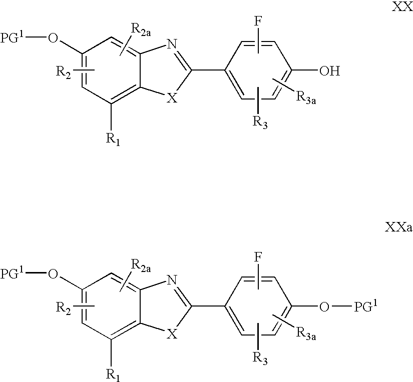

or salts thereof, wherein R₁, R₂, R₂ₐ, R₃, R₃ₐ, PG¹ and X are the same as defined in the compounds Formula II herein. As used herein, the term “substantially free of compounds of Formula XX or XXa” means that no more than about 5% by weight, preferably no more than about 2% by weight, more preferably no more that about 1% by weight, and more preferably no more than about 0.5% by weight of a given sample of compound has Formula XX or XXa or a salt thereof. In some embodiments, less than about one molar equivalent of the base (relative to the amount of the compound of Formula II) is used to minimize deprotonation of the hydroxyl attached to the fused benzene ring of the bi-cyclic ring of the compound of Formula III. In some embodiments, less than 1.05, 1.02, 1.01, 1.0, 0.99, or 0.98 molar equivalent of the base (relative to the amount of the compound of Formula III) is used. In some embodiments, about one molar equivalent of the base (relative to the amount of the compound of Formula III) is used. In some embodiments, about 0.95 to about 1.02 molar equivalent of the base (relative to the amount of the compound of Formula II) is used. In some embodiments, about 0.95 to about 1.02 molar equivalent of the base (relative to the amount of the compound of Formula III) is used. In some embodiments, about 0.98 to about 1.01 molar equivalent of the base (relative to the amount of the compound of Formula III) is used. In some embodiments, about 0.98 to about 1.00 molar equivalent of the base (relative to the amount of the compound of Formula II) is used. In some embodiments, less than about one molar equivalent of the hydroxyl protecting group reagent (relative to the amount of the compound of Formula III) is used in the reaction to minimize the formation of compound of Formula XXa (protecting both hydroxyl groups of the compound Formula II). In some embodiments, about 0.98 to about 1.01 molar equivalent of the hydroxyl protecting group reagent (relative to the amount of the compound of Formula II) is used. In some embodiments, about 0.98 to about 1.0 molar equivalent of the hydroxyl protecting group reagent (relative to the amount of the compound of Formula II) is used. In some embodiments, about one molar equivalent of the hydroxyl protecting group reagent (relative to the amount of the compound of Formula II) is used.

As seen in Scheme I above, the protected compound of Formula II is then reacted with a sulfating reagent. This reaction can produce a compound of Formula Ia or a salt thereof, as shown in Scheme 1. In some embodiments, during the workup of isolating the produced compound of Formula Ia or salt thereof from the reaction mixture, the workup conditions are sufficient to remove the protecting group —PG¹ of the compound of Formula Ia or a salt thereof to afford the compound of Formula I or a salt thereof. Several sulfating reagents are known for sulfation of hydroxyl groups, including aromatic hydroxyl groups. In some embodiments, the sulfating reagent is a complex of sulfur trioxide and an amide, for example, a complex of sulfur trioxide and N,N-dimethylformamide. In one preferred embodiment, the sulfating reagent is a complex of sulfur trioxide and an amine, for example a tertiary amine [including acyclic amines (for example, trimethylamine, triethylamine, dimethylphenylamine and dimethylbenzylamine), cyclic amines (for example, 1-methylpyrrolidine and 1-methylpiperidine) and aromatic amines which have one or more nitrogen atoms as ring-forming atoms of the aromatic ring, for example, 1-methylimidazole, pyridine and pyrimidine]. In some embodiments, the sulfating reagent is a complex of sulfur trioxide and aromatic amine. In some embodiments, the sulfating reagent is a sulfur trioxide/pyridine complex. Other complexes of sulfur trioxide and a tertiary amine, for example, sulfur trioxide and trimethylamine complex or sulfur trioxide and triethylamine complex, can also be used as sulfating reagents.

Generally, the sulfating reagent is employed in molar excess relative to the amount of compound of Formula II or salt thereof. For example, the ratio of the sulfating reagent to the compound of Formula II or the salt thereof can be a value of between about 1 and about 2, for example about 1.4 to about 1.6.

Typically, the reaction of the compound of Formula II and the sulfating reagent is performed in a solvent system that includes a solvent or mixture of solvents. A wide variety of suitable solvents can be employed, including polar organic solvents, preferably polar aprotic organic solvents, including those describe above. In some embodiments, the reaction is performed in a solvent system that includes or consists of acetonitrile.

The reaction of the compound of Formula II and the sulfating reagent is performed at convenient temperature, for example from about 20° C. to about 60° C., preferably at from about 40° C. to about 50° C. Generally, the reaction temperature can be raised to accelerate the rate of the reaction. In some embodiments, the reaction mixture is heated to reflux in the solvent system of the reaction. Typically, the compound of Formula II is dissolved in solvent, and the sulfating agent is added slowly. The progress of the reaction can be monitored by a variety of techniques, for example by chromatographic techniques. The reaction between the compound of Formula II and the sulfating reagent is complete after about 12 hours to about 2 days. It is advantageous to collect the sulfated product of Formula I as the sulfate salt, to prevent loss of the relatively labile sulfate group during workup and purification. Thus, in some embodiments, when the reaction between the compound of Formula II and the sulfating reagent is complete, the reaction mixture is treated with a base, preferably an inorganic base, for example a group I or group II metal bicarbonate (such as NaHCO₃ and KHCO₃), and the compound of Formula I is isolated as the sulfate salt. In some embodiments, the isolated salt has Formula Ib:

[R¹⁰—O—SO₃⁻¹]_{q}M Ib

wherein:

R¹⁰ is:

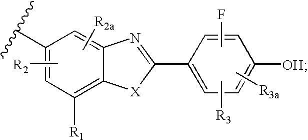

M is a Group I or II metal ion; and

q is 1 when M is Group I metal ion, or q is 2 when M is a Group II metal ion.

In some preferred embodiments, R¹⁰ has Formula R^{10a}:

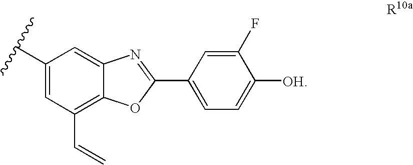

In some such embodiments, M is Na⁺ ion.

The salt can be isolated form the reaction mixture by applying standard techniques, for example distillation; distillation under reduced pressure; distillation further facilitated by adding a co-solvent; distillation under reduced pressure further facilitated by adding a co-solvent; or triturating the salt with an organic solvent system (an organic solvent system include one or more organic solvents), for example one or more polar organic solvents. For example, in some embodiments, after the reaction between the compound of Formula II and the sulfating reagent is complete, the reaction mixture is poured into aqueous base, for example an aqueous 10% sodium bicarbonate solution, and the compound of Formula I is isolated as the sulfate salt, for example the sodium sulfate salt by evaporating the residue, coevaporating with acetonitrile, and triturating the residue with one or more solvents, for example methanol, ethanol or 2-butanone.

Generally, the workup conditions are sufficient to remove the protecting group —PG¹ of the compound of Formula Ia or the salt thereof to afford the compound of Formula I or a salt thereof directly. However, in embodiments where workup conditions are not sufficient to remove the hydroxyl protecting group —PG¹ of the compound of formula Ia or the salt thereof, the processes of the invention include the further step of removing the hydroxyl protecting group —PG¹. Choice of conditions effective to remove the protecting group will vary depending on the specific protecting group employed, and include for example for treatment with acid or base. In some embodiments, where the hydroxyl protecting group is tert-butyldimethylsilyl (TBS), the TBS group can be removed by reaction with a fluoride salt, for example an tetraalkylammonium salt, such as tetrabutylammonium fluoride. In some embodiments, the yield of the compound of Formula I or salt thereof (from the compound of Formula II or salt thereof) is greater than 45%, 50%, 55%, 60%, 65%, 75%, 80%, or 85%. In some embodiments, the yield of the compound of Formula I or salt thereof (from the compound of Formula II or salt thereof) is greater than 55%, 60%, 65%, 75%, 80%, or 85%.

In a further aspect, the present invention provides a process comprising steps (a), (b), (c), (d), and (e) for the selective preparation of a mono-sulfated compound of Formula I:

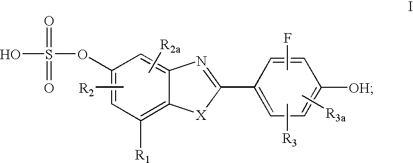

or salt thereof, comprising:

(a) reacting a compound of Formula III:

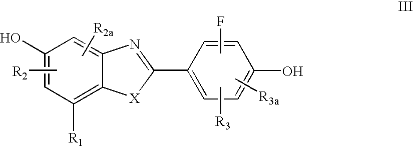

or salt thereof in a polar aprotic solvent with a protecting group reagent PG¹-Q wherein Q is a leaving group in the presence of a base wherein the amount of the base is from about 0.95 to about 1.02 molar equivalents relative to the compound of Formula III and the amount of the protecting group reagent is at least one molar equivalent relative to the compound of Formula II, and wherein said reaction forms a compound of Formula II:

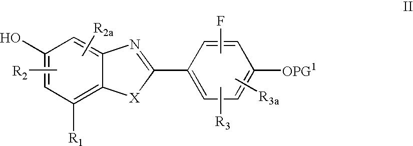

or salt thereof;

(b) optionally isolating the compound of Formula II or salt thereof from the reaction mixture and optionally purifying said isolated compound or salt thereof;

(c) reacting the compound of Formula II or salt thereof with a sulfating reagent in the amount of from about 1.0 to about 2.0 molar equivalents relative to the amount of the compound of Formula II or salt thereof, in the presence of a polar aprotic solvent to form a compound of Formula I or Ia:

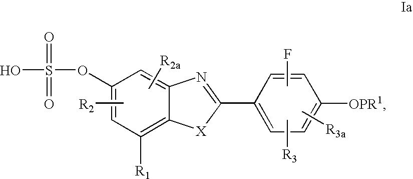

a salt thereof or a mixture thereof;

(d) treating the reaction mixture from step (c) with an inorganic base of a metal of Group I or II to form a salt of Formula Ib and additionally at least partially converting any compound of Formula Ia or salt thereof to the salt of Formula Ib:

[R¹⁰—O—SO₃⁻¹]_{q}M Ib

wherein:

R¹⁰ is:

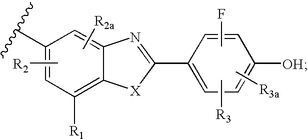

M is a Group I or II metal ion; and

q is 1 when M is Group I metal ion, or q is 2 when M is a Group II metal ion; and

(e) if necessary removing the PG¹ group of any compound of Formula Ia or salt thereof, if present, to convert it to the compound of Formula I or salt thereof,

wherein:
PG¹ is a hydroxyl protecting group;
R₁ is hydrogen, hydroxyl, halogen, alkyl of 1-6 carbon atoms, trifluoroalkyl of 1-6 carbon atoms, cycloalkyl of 3-8 carbon atoms, alkoxy of 1-6 carbon atoms, trifluoroalkoxy of 1-6 carbon atoms, thioalkyl of 1-6 carbon atoms, sulfoxoalkyl of 1-6 carbon atoms, sulfonoalkyl of 1-6 carbon atoms, aryl of 6-10 carbon atoms, a 5 or 6-membered heterocyclic ring having 1 to 4 heteroatoms selected from O, N or S, —NO₂, —NR₅R₆, —N(R₅)COR₆, —CN, —CHFCN, —CF₂CN, alkynyl of 2-7 carbon atoms, or alkenyl of 2-7 carbon atoms; wherein the alkyl or alkenyl moieties are optionally substituted with hydroxyl, —CN, halogen, trifluoroalkyl, trifluoroalkoxy, —COR₅, —CO₂R₅, —NO₂, CONR₅R₆, NR₅R₆ or N(R₅)COR₆;
R₂ and R₂ₐ are each, independently, hydrogen, hydroxyl, halogen, alkyl of 1-6 carbon atoms, alkoxy of 1-4 carbon atoms, alkenyl of 2-7 carbon atoms, or alkynyl of 2-7 carbon atoms, trifluoroalkyl of 1-6 carbon atoms, or trifluoroalkoxy of 1-6 carbon atoms; wherein the alkyl or alkenyl moieties are optionally substituted with hydroxyl, —CN, halogen, trifluoroalkyl, trifluoroalkoxy, —COR₅, —CO₂R₅, —NO₂, CONR₅R₆, NR₅R₆ or N(R₅)COR₆;
R₃, R₃ₐ, and R₄ are each, independently, hydrogen, alkyl of 1-6 carbon atoms, alkenyl of 2-7 carbon atoms, alkynyl of 2-7 carbon atoms, halogen, alkoxy of 1-4 carbon atoms, trifluoroalkyl of 1-6 carbon atoms, or trifluoroalkoxy of 1-6 carbon atoms; wherein the alkyl or alkenyl moieties are optionally substituted with hydroxyl, —CN, halogen, trifluoroalkyl, trifluoroalkoxy, —COR₅, —CO₂R₅, —NO₂, CONR₅R₆, NR₅R₆ or N(R₅)COR₆;
R₅, R₆ are each, independently hydrogen, alkyl of 1-6 carbon atoms, aryl of 6-10 carbon atoms;
X is O, S, or NR₇; and
R₇ is hydrogen, alkyl of 1-6 carbon atoms, aryl of 6-10 carbon atoms, —COR₅, —CO₂R₅ or —SO₂R₅.

It is appreciated that certain features of the invention, which are, for clarity, described in the context of separate embodiments, can also be provided in combination in a single embodiment. Conversely, various features of the invention which are, for brevity, described in the context of a single embodiment, can also be provided separately or in any suitable subcombination.

The term “alkyl”, employed alone, is defined herein as, unless otherwise stated, either a straight-chain or branched saturated hydrocarbon moiety. In some embodiments, the alkyl moiety contains 1 to 18, 1 to 12, 1 to 10, 1 to 8, 1 to 6, or 1 to 4 carbon atoms. Examples of saturated hydrocarbon alkyl moieties include, but are not limited to, chemical groups such as methyl, ethyl, n-propyl, isopropyl, n-butyl, tert-butyl, isobutyl, sec-butyl; higher homologs such as n-pentyl, n-hexyl, n-heptyl, n-octyl, and the like.

The term “alkylenyl” refers to a bivalent straight-chained or branched alkyl group.

As used herein, “alkenyl” refers to an alkyl group having one or more carbon-carbon double bonds. Nonlimiting examples of alkenyl groups include ethenyl, propenyl, and the like.

As used herein, “alkynyl” refers to an alkyl group having one or more carbon-carbon triple bonds. Nonlimiting examples of alkynyl groups include ethynyl, propynyl, and the like.

The term “alkoxy”, employed alone or in combination with other terms, is defined herein as, unless otherwise stated, —O-alkyl. Examples of alkoxy moieties include, but are not limited to, chemical groups such as methoxy, ethoxy, isopropoxy, sec-butoxy, tert-butoxy, and the like.

The term “cycloalkyl”, employed alone or in combination with other terms, is defined herein as, unless otherwise stated, a monocyclic, bicyclic, tricyclic, fused, bridged, or spiro monovalent non-aromatic hydrocarbon moiety of 3-18 or 3-7 carbon atoms. Also included in the definition of cycloalkyl are moieties that have one or more aromatic rings fused (i.e., having a bond in common with) to the nonaromatic ring. Any suitable ring position of the cycloalkyl moiety can be covalently linked to the defined chemical structure. Examples of cycloalkyl moieties include, but are not limited to, chemical groups such as cyclopropyl, cyclobutyl, cyclopentyl, cyclohexyl, cycloheptyl, norbornyl, adamantyl, spiro[4.5]decanyl, and the like.

The terms “halo” or “halogen”, employed alone or in combination with other terms, is defined herein as, unless otherwise stated, fluoro, chloro, bromo, or iodo.

As used herein, the term “reacting” refers to the bringing together of designated chemical reactants such that a chemical transformation takes place generating a compound different from any initially introduced into the system. Reacting can take place in the presence or absence of solvent.

The compounds of the present invention can contain an asymmetric atom, and some of the compounds can contain one or more asymmetric atoms or centers, which can thus give rise to optical isomers (enantiomers) and diastereomers. The present invention includes such optical isomers (enantiomers) and diastereomers (geometric isomers), as well as, the racemic and resolved, enantiomerically pure R and S stereoisomers, as well as, other mixtures of the R and S stereoisomers and pharmaceutically acceptable salts thereof. Optical isomers can be obtained in pure form by standard procedures known to those skilled in the art, and include, but are not limited to, diastereomeric salt formation, kinetic resolution, and asymmetric synthesis. It is also understood that this invention encompasses all possible regioisomers, and mixtures thereof, which can be obtained in pure form by standard separation procedures known to those skilled in the art, and include, but are not limited to, column chromatography, thin-layer chromatography, and high-performance liquid chromatography.

Compounds of the invention can also include all isotopes of atoms occurring in the intermediates or final compounds. Isotopes include those atoms having the same atomic number but different mass numbers. For example, isotopes of hydrogen include tritium and deuterium.

Compounds of the invention can also include tautomeric forms, such as keto-enol tautomers. Tautomeric forms can be in equilibrium or sterically locked into one form by appropriate substitution.

The processes described herein can be monitored according to any suitable method known in the art. For example, product formation can be monitored by spectroscopic means, such as nuclear magnetic resonance spectroscopy (e.g., ¹H or ¹³C), infrared spectroscopy, spectrophotometry (e.g., UV-visible), or mass spectrometry, or by chromatography such as high performance liquid chromatography (HPLC) or thin layer chromatography.

The reactions of the processes described herein can be carried out in air or under an inert atmosphere. Typically, reactions containing reagents or products that are substantially reactive with air can be carried out using air-sensitive synthetic techniques that are well known to the skilled artisan.

Upon carrying out preparation of compounds according to the processes described herein, the usual isolation and purification operations such as concentration, filtration, extraction, solid-phase extraction, recrystallization, chromatography, and the like may be used to isolate the desired products.

The invention will be described in greater detail by way of specific examples. The following examples are offered for illustrative purposes, and are not intended to limit the invention in any manner. Those of skill in the art will readily recognize a variety of noncritical parameters which can be changed or modified to yield essentially the same results.

### EXAMPLES

### Example 1

### Preparation of sulfuric acid mono-[2-(3-fluoro-4-hydroxy-phenyl)-7-vinyl-benzooxazol-5-yl]ester sodium salt

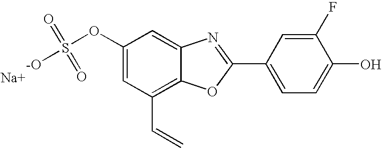

The title compound was prepared by selective TBS-protection of 2-(3-Fluoro-4-hydroxyphenyl)-7-vinylbenzooxazol-5-ol at the fluorophenol hydroxyl group (determined by NMR), using TBSCl/imidazole. Upon treatment of the product with pyridine-SO₃ complex and acidification with DOWEX-H⁺ and freeze-drying, the starting material was obtained, indicating lability of the sulphated phenol. Accordingly, the more stable sodium sulphate was prepared by the following procedure.

### A. Preparation of 2-(3-Fluoro-4-tert-butyldimethylsilyloxyphenyl)-7-vinylbenzooxazol-5-ol

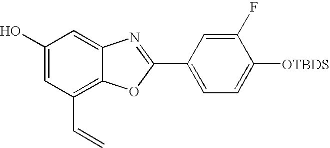

2-(3-Fluoro-4-hydroxyphenyl)-7-vinylbenzooxazol-5-ol (5 g, 18 mmol; prepared as described in U.S. Pat. No. 6,794,403, incorporated by reference herein in its entirety) was dissolved in 50 mL of tetrahydrofuran (THF). Imidazole (1.2 g, 18 mmol) was added, and the solution was cooled to 0° C. A solution of t-butyldimethylsilyl chloride (2.7 g, 18 mmol) in THF (20 mL) was added dropwise over 10 minutes. After 18 hours reaction at room temperature, the mixture was filtered and evaporated. The residue was purified with flash chromatography over silica (using ethylacetate/heptane) to yield 4 g (58%) of 2-(3-Fluoro-4-tert-butyldimethylsilyloxyphenyl)-7-vinylbenzooxazol-5-ol as a white solid.

### B. Preparation of sulfuric acid mono-[2-(3-fluoro-4-hydroxy-phenyl)-7-vinyl-benzooxazol-5-yl]ester sodium salt

2-(3-Fluoro-4-tert-butyldimethylsilyloxyphenyl)-7-vinylbenzooxazol-5-ol (2 g, 5.2 mmol) was dissolved in 60 mL of acetonitrile at 43° C. Pyridine.SO₃ (50%) (2.5 g, 8 mmol) was added in 5 portions over 1.5 hours. The solution was stirred overnight at room temperature and poured into 40 mL of 10% NaHCO₃. The water/acetonitrile was evaporated (with foaming noted) and the residue coevaporated with acetonitrile. The residue was triturated with methanol, filtered and dried. A sample of 1 g of the residue was triturated with 2-butanone and filtered. The solids were triturated with ethanol at 50° C. The mixture was filtered and the ethanol evaporated to yield 0.58 g (61%) of 2-(3-Fluoro-4-sulfatephenyl)-7-vinylbenzooxazol-5-ol sodium salt as an off-white solid (the TBS-group was simultaneously removed under the applied conditions). ¹H NMR on the product conformed to the assigned structure. MW determined to be 373.3. HPLC-MS: M−1=350.

Those skilled in the art will recognize that various changes and/or modifications may be made to aspects or embodiments of this invention and that such changes and/or modifications may be made without departing from the spirit of this invention. Therefore, it is intended that the appended claims cover all such equivalent variations as will fall within the spirit and scope of this invention.

It is intended that each of the patents, applications, and printed publications, including books, mentioned in this patent document be hereby incorporated by reference in their entirety.

What is claimed is:

1. A synthetic process comprising:
reacting a compound of Formula II: 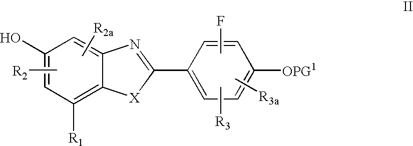
or salt thereof, wherein:
PG¹ is a hydroxyl protecting group;
R₁ is hydrogen, hydroxyl, halogen, alkyl of 1-6 carbon atoms, trifluoroalkyl of 1-6 carbon atoms, cycloalkyl of 3-8 carbon atoms, alkoxy of 1-6 carbon atoms, trifluoroalkoxy of 1-6 carbon atoms, thioalkyl of 1-6 carbon atoms, sulfoxoalkyl of 1-6 carbon atoms, sulfonoalkyl of 1-6 carbon atoms, aryl of 6-10 carbon atoms, a 5 or 6-membered heterocyclic ring having 1 to 4 heteroatoms selected from O, N or S, —NO₂, —NR₅R₆, —N(R₅)COR₆, —CN, —CHFCN, —CF₂CN, alkynyl of 2-7 carbon atoms, or alkenyl of 2-7 carbon atoms; wherein the alkyl or alkenyl moieties are optionally substituted with hydroxyl, —CN, halogen, trifluoroalkyl, trifluoroalkoxy, —COR₅, —CO₂R₅, —NO₂, CONR₅R₆, NR₅R₆ or N(R₅)COR₆;
R₂ and R₂ₐ are each, independently, hydrogen, hydroxyl, halogen, alkyl of 1-6 carbon atoms, alkoxy of 1-4 carbon atoms, alkenyl of 2-7 carbon atoms, or alkynyl of 2-7 carbon atoms, trifluoroalkyl of 1-6 carbon atoms, or trifluoroalkoxy of 1-6 carbon atoms; wherein the alkyl or alkenyl moieties are optionally substituted with hydroxyl, —CN, halogen, trifluoroalkyl, trifluoroalkoxy, —COR₅, —CO₂R₅, —NO₂, CONR₅R₆, NR₅R₆ or N(R₅)COR₆;
R₃, R₃ₐ, and R₄ are each, independently, hydrogen, alkyl of 1-6 carbon atoms, alkenyl of 2-7 carbon atoms, alkynyl of 2-7 carbon atoms, halogen, alkoxy of 1-4 carbon atoms, trifluoroalkyl of 1-6 carbon atoms, or trifluoroalkoxy of 1-6 carbon atoms; wherein the alkyl or alkenyl moieties are optionally substituted with hydroxyl, —CN, halogen, trifluoroalkyl, trifluoroalkoxy, —COR₅, —CO₂R₅, —NO₂, CONR₅R₆, NR₅R₆ or N(R₅)COR₆;
R₅, R₆ are each, independently hydrogen, alkyl of 1-6 carbon atoms, aryl of 6-10 carbon atoms;
X is O, S, or NR₇; and
R₇ is hydrogen, alkyl of 1-6 carbon atoms, aryl of 6-10 carbon atoms, —COR₅, —CO₂R₅ or —SO₂R₅,
with a sulfating reagent, for a time and under conditions sufficient to form a compound of Formula I or Ia: 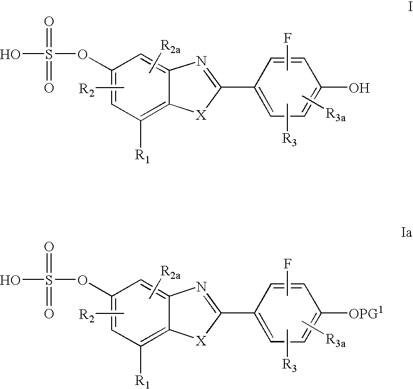
or salt thereof, or a mixture thereof.

2. The process of claim 1, wherein the compound of Formula II has the structure of Formula II-1: 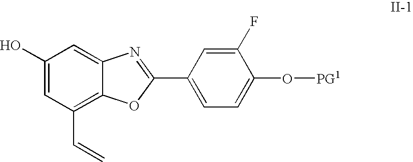
or is a salt thereof;
the compound of Formula I has the structure of Formula I-1: 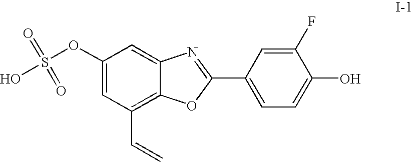
or is a salt thereof; and
the compound of Formula Ia has the structure of Formula Ia-1: 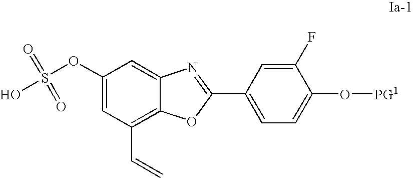
or is a salt thereof.

3. The process of claim 1, wherein PG¹ is —SiR^{a}R^{b}R^{c}; wherein R^{a}R^{b} and R^{c} are each, independently, C₁₋₆ alkyl.

4. The process of claim 1, wherein PG¹ is tert-butyldimethylsilyl.

5. The process of claim 1, wherein the sulfating reagent comprises a complex of sulfur trioxide and a tertiary amine.

6. The process of claim 1, wherein the sulfating reagent comprises sulfur trioxide pyridine complex.

7. The process of claim 1, wherein the molar ratio of the sulfating reagent to the compound of Formula II or salt thereof is a value of between about 1.0 and about 2.0.

8. The process of claim 1, wherein the sulfating is performed in a solvent system comprising a polar, aprotic organic solvent.

9. The process of claim 8, wherein the solvent system comprises acetonitrile.

10. The process of claim 8, further comprising contacting the compound of Formula I or Ia, or salt thereof, in the solvent system, with an inorganic base.

11. The process of claim 10, wherein the inorganic base is selected from NaHCO₃ and KHCO₃.

12. The process of claim 1, further comprising isolating the compound of Formula Ia or salt thereof.

13. The process of claim 1, further comprising removing the PG¹ group of the compound of Formula Ia or the salt thereof to form the compound of Formula I or salt thereof.

14. The process of claim 1, further comprising isolating a salt of the compound of Formula I, wherein the salt has Formula Ib:
[R¹⁰—O—SO₃⁻¹]_{q}M Ib
wherein:
R¹⁰ is: 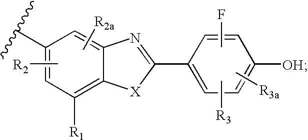
M is a Group I or II metal ion; and
q is 1 when M is Group I metal ion, or q is 2 when M is a Group II metal ion.

15. The process of claim 14, wherein R¹⁰ has Formula R^{10a}:

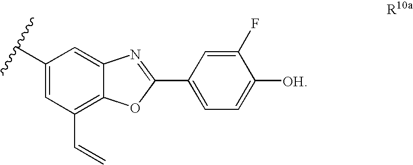

16. The process of claim 15 wherein M is Na⁺ ion.

17. The process of claim 15, wherein the isolating the salt of the compound of Formula I comprises one or more of distillation; distillation under reduced pressure; distillation further facilitated by adding a co-solvent; distillation under reduced pressure further facilitated by adding a co-solvent; or triturating the salt with an organic solvent system, and wherein the organic solvent system comprises a polar organic solvent.

18. The process of claim 17, wherein the organic solvent system comprises methanol, ethanol or 2-butanone.

19. The process of claim 1 further comprising reacting a compound of Formula III: 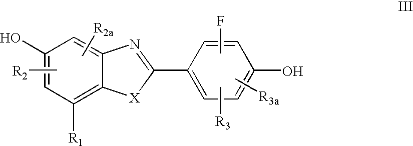
or salt thereof, with a hydroxyl protecting group reagent for a time and under conditions sufficient to form the compound of Formula II or salt thereof.

20. The process of claim 2 further comprising reacting a compound of Formula IIIa: 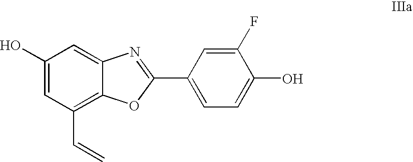
or salt thereof, with a hydroxyl protecting group reagent for a time and under conditions sufficient to form the compound of Formula II-1 or salt thereof.

21. The process of claim 20, wherein the amount of the hydroxyl protecting group reagent is about one molar equivalent relative to the amount of the compound of Formula IIa or salt thereof.

22. The process of claim 20, wherein the hydroxyl protecting group reagent has the formula PG¹-Q; and Q is a leaving group.

23. The process of claim 22 wherein PG¹ is —SiR^{a}R^{b}R^{c}; and R^{a}, R^{b} and R^{c} are each, independently, C₁₋₆ alkyl.

24. The process of claim 20, wherein the reaction of the compound of Formula IIIa or salt thereof with the hydroxyl protecting group reagent is performed in the presence of a base, and wherein the amount of the base is about one molar equivalent relative to the amount of the compound of Formula IIIa or salt thereof.

25. The process of claim 20, further comprising isolating the formed compound of Formula II-1 or salt thereof.

26. A compound of Formula II: 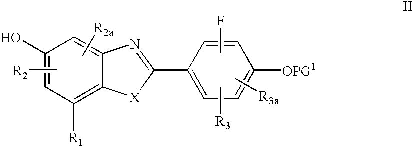
or salt thereof, wherein:
PG¹ is a hydroxyl protecting group;
R₁ is hydrogen, hydroxyl, halogen, alkyl of 1-6 carbon atoms, trifluoroalkyl of 1-6 carbon atoms, cycloalkyl of 3-8 carbon atoms, alkoxy of 1-6 carbon atoms, trifluoroalkoxy of 1-6 carbon atoms, thioalkyl of 1-6 carbon atoms, sulfoxoalkyl of 1-6 carbon atoms, sulfonoalkyl of 1-6 carbon atoms, aryl of 6-10 carbon atoms, a 5 or 6-membered heterocyclic ring having 1 to 4 heteroatoms selected from O, N or S, —NO₂, —NR₅R₆, —N(R₅)COR₆, —CN, —CHFCN, —CF₂CN, alkynyl of 2-7 carbon atoms, or alkenyl of 2-7 carbon atoms; wherein the alkyl or alkenyl moieties are optionally substituted with hydroxyl, —CN, halogen, trifluoroalkyl, trifluoroalkoxy, —COR₅, —CO₂R₅, —NO₂, CONR₅R₆, NR₅R₆ or N(R₅)COR₆;
R₂ and R₂ₐ are each, independently, hydrogen, hydroxyl, halogen, alkyl of 1-6 carbon atoms, alkoxy of 1-4 carbon atoms, alkenyl of 2-7 carbon atoms, or alkynyl of 2-7 carbon atoms, trifluoroalkyl of 1-6 carbon atoms, or trifluoroalkoxy of 1-6 carbon atoms; wherein the alkyl or alkenyl moieties are optionally substituted with hydroxyl, —CN, halogen, trifluoroalkyl, trifluoroalkoxy, —COR₅, —CO₂R₅, —NO₂, CONR₅R₆, NR₅R₆ or N(R₅)COR₆;
R₃, R₃ₐ, and R₄ are each, independently, hydrogen, alkyl of 1-6 carbon atoms, alkenyl of 2-7 carbon atoms, alkynyl of 2-7 carbon atoms, halogen, alkoxy of 1-4 carbon atoms, trifluoroalkyl of 1-6 carbon atoms, or trifluoroalkoxy of 1-6 carbon atoms; wherein the alkyl or alkenyl moieties are optionally substituted with hydroxyl, —CN, halogen, trifluoroalkyl, trifluoroalkoxy, —COR₅, —CO₂R₅, —NO₂, CONR₅R₆, NR₅R₆ or N(R₅)COR₆;
R₅, R₆ are each, independently hydrogen, alkyl of 1-6 carbon atoms, aryl of 6-10 carbon atoms;
X is O, S, or NR₇; and
R₇ is hydrogen, alkyl of 1-6 carbon atoms, aryl of 6-10 carbon atoms, —COR₅, —CO₂R₅ or —SO₂R₅.

27. The compound or salt thereof of claim 26, wherein the compound has the structure of Formula II-1:

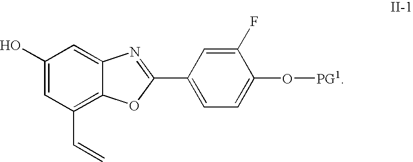

28. A compound of Formula Ia: 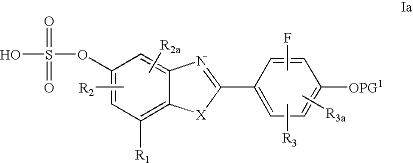
or salt thereof, wherein:
PG¹ is a hydroxyl protecting group;
R₁ is hydrogen, hydroxyl, halogen, alkyl of 1-6 carbon atoms, trifluoroalkyl of 1-6 carbon atoms, cycloalkyl of 3-8 carbon atoms, alkoxy of 1-6 carbon atoms, trifluoroalkoxy of 1-6 carbon atoms, thioalkyl of 1-6 carbon atoms, sulfoxoalkyl of 1-6 carbon atoms, sulfonoalkyl of 1-6 carbon atoms, aryl of 6-10 carbon atoms, a 5 or 6-membered heterocyclic ring having 1 to 4 heteroatoms selected from O, N or S, —NO₂, —NR₅R₆, —N(R₅)COR₆, —CN, —CHFCN, —CF₂CN, alkynyl of 2-7 carbon atoms, or alkenyl of 2-7 carbon atoms; wherein the alkyl or alkenyl moieties are optionally substituted with hydroxyl, —CN, halogen, trifluoroalkyl, trifluoroalkoxy, —COR₅, —CO₂R₅, —NO₂, CONR₅R₆, NR₅R₆ or N(R₅)COR₆;
R₂ and R₂ₐ are each, independently, hydrogen, hydroxyl, halogen, alkyl of 1-6 carbon atoms, alkoxy of 1-4 carbon atoms, alkenyl of 2-7 carbon atoms, or alkynyl of 2-7 carbon atoms, trifluoroalkyl of 1-6 carbon atoms, or trifluoroalkoxy of 1-6 carbon atoms; wherein the alkyl or alkenyl moieties are optionally substituted with hydroxyl, —CN, halogen, trifluoroalkyl, trifluoroalkoxy, —COR₅, —CO₂R₅, —NO₂, CONR₅R₆, NR₅R₆ or N(R₅)COR₆;
R₃, R₃ₐ, and R₄ are each, independently, hydrogen, alkyl of 1-6 carbon atoms, alkenyl of 2-7 carbon atoms, alkynyl of 2-7 carbon atoms, halogen, alkoxy of 1-4 carbon atoms, trifluoroalkyl of 1-6 carbon atoms, or trifluoroalkoxy of 1-6 carbon atoms; wherein the alkyl or alkenyl moieties are optionally substituted with hydroxyl, —CN, halogen, trifluoroalkyl, trifluoroalkoxy, —COR₅, —CO₂R₅, —NO₂, CONR₅R₆, NR₅R₆ or N(R₅)COR₆;
R₅, R₆ are each, independently hydrogen, alkyl of 1-6 carbon atoms, aryl of 6-10 carbon atoms;
X is O, S, or NR₇; and
R₇ is hydrogen, alkyl of 1-6 carbon atoms, aryl of 6-10 carbon atoms, —COR₅, —CO₂R₅ or —SO₂R₅.

29. The compound or salt thereof of claim 28, wherein the compound has the structure of Formula Ia-1: [chemical structure]
